(19) United States
(12) Patent Application Publication
(10) Pub. No.: US 2020/0261049 A1
(43) Pub. Date: Aug. 20, 2020

(54) MEDICAL IMAGING APPARATUS AND METHOD OF CONTROLLING THE SAME

(71) Applicant: Samsung Electronics Co., Ltd., Suwon-si (KR)

(72) Inventors: Jongjyh HSIEH, Suwon-si (KR); Minkook CHO, Suwon-si (KR)

(73) Assignee: Samsung Electronics Co., Ltd., Suwon-si (KR)

(21) Appl. No.: 16/793,108

(22) Filed: Feb. 18, 2020

(30) Foreign Application Priority Data
Feb. 15, 2019 (KR) 10-2019-0017720

(51) Int. Cl. A61B 6/00; F16M 11/18; F16M 11/10; F16M 11/22; F16M 11/42; F16F 9/53; F16F 9/50

(52) U.S. Cl. CPC A61B 6/547; A61B 6/4405; F16M 11/18; F16M 11/105; A61B 6/035; F16M 11/42; F16F 9/532; F16F 9/535; F16F 9/50; F16M 11/22

(57) ABSTRACT

The disclosure relates to a medical imaging apparatus, and more particularly, to a mobile computed tomography apparatus for generating X-ray tomography images, and a method of controlling the mobile computed tomography apparatus. The medical imaging apparatus includes a gantry that is moveable; a moving device configured to move the medical imaging apparatus that includes the gantry; a damper configured to attenuate vibration generated by a movement of the gantry or a movement of the medical imaging apparatus; an inputter configured to receive an image capturing protocol for an object; and a controller configured to determine a coefficient of the damper for attenuating the vibration generated by the movement of the gantry or the movement of the medical imaging apparatus corresponding to the input image capturing protocol, and to apply a current to the damper to operate the damper according to the determined coefficient of the damper.

### CROSS-REFERENCE TO RELATED APPLICATION(S)

This application is based on and claims priority under 35 U.S.C. § 119 to Korean Patent Application No. 10-2019-0017720, filed on Feb. 15, 2019 in the Korean Intellectual Property Office, the disclosure of which is incorporated by reference in its entirety.

### FIELD

The disclosure relates to a medical imaging apparatus, and more particularly, to a mobile computed tomography apparatus for generating X-ray tomography images, and a method of controlling the mobile computed tomography apparatus.

### DESCRIPTION OF RELATED ART

Medical imaging apparatuses are used to obtain images of an internal structure of an object. A medical image processing apparatus, as a non-invasive examination apparatus, obtains images of structural details of internal tissues, fluid streams, and the like of a human body, processes the obtained images, and displays the processed image to users. The users such as doctors may diagnose physical conditions and diseases of patients by using medical images output from the medical image processing apparatus.

A computed tomography (CT) apparatus is a representative imaging apparatus used to obtain an image of the object via irradiation of X-rays.

The CT apparatus may provide an image of the object, i.e., an internal structure (e.g., an organ such as a kidney and a lung) of the object without an overlap therebetween. Thus, the CT apparatus has been widely used for accurate diagnosis of diseases. Hereinafter, the medical image obtained by a tomographic apparatus will be referred to as a tomogram.

A general tomographic apparatus performs tomography while a table on which the object is located has been inserted into the tomographic apparatus. The tomographic apparatus may reconstruct the tomogram of the object by using data obtained by the tomography.

Recently, in order to improve convenience and usability of the tomographic apparatus, a mobile CT apparatus capable of moving the tomographic apparatus to the object is being developed. In the mobile CT apparatus, the tomographic apparatus moves directly to the object where movement of the object is inconvenient, and the tomographic apparatus may perform the tomography while scanning the object instead of the table on which the object is located.

However, unlike a fixed tomographic apparatus, the mobile CT apparatus does not have a fixed position of a gantry, and thus vibrations are caused by rotation of the gantry and movement of the mobile CT apparatus. When a surface of a floor where the tomography is performed is uneven, the shaking is further amplified when the mobile CT apparatus is moved, which may cause a noise in a tomographic image.

### SUMMARY

In accordance with an aspect of the disclosure, a medical imaging apparatus includes a gantry provided to rotate; a moving device configured to move the medical imaging apparatus provided with the gantry; a damper configured to attenuate vibration generated by movement of the gantry or movement of the medical imaging apparatus; an inputter configured to receive an image capturing protocol for an object; and a controller configured to determine a damping coefficient of the damper for attenuating the vibration generated by the movement of the gantry or the movement of the medical imaging apparatus corresponding to the input image capturing protocol, and to apply a current to the damper to operate the damper according to the determined damping coefficient.

The medical imaging apparatus may further include a storage configured to store data about the damping coefficient predetermined according to the image capturing protocol. The predetermined damping coefficient may include at least one of a damping coefficient for attenuating the vibration generated by rotation of the gantry and a damping coefficient for attenuating the vibration generated as the gantry moves in a direction of scanning the object.

The gantry may include a rotating frame configured to rotate according to a predetermined rotation axis. The damper may be provided on the rotating frame.

The moving device may include a wheel configured to move the medical imaging apparatus provided with the gantry in a direction of scanning the object. The damper may be provided on the wheel.

The damper may be provided in plurality in the rotating frame. The plurality of dampers may be spaced apart from each other according to a predetermined distance on the rotating frame.

The wheel may be provided in plurality. The damper may be provided in plurality in correspondence with each of the plurality of wheels.

The controller may be configured to apply the current to the damper to change the amount of fluid flowing in the damper or to change the viscosity of the fluid for operating the damper according to the determined damping coefficient.

The damper may include a piston channel through which the fluid flows. The controller may be configured to control the opening and closing of the piston channel by applying the current to the damper to change the amount of fluid flowing in the damper.

The damper may include an electromagnet configured to form a magnetic field in response to the application of the current. The controller may be configured to change the viscosity of the fluid by applying the current to the electromagnet to form a magnetic field inside the damper.

The medical imaging apparatus may further include an X-ray source provided inside the gantry, and configured to irradiate X-rays; and a sensing device configured to obtain vibration data generated by the movement of the gantry or the movement of the medical imaging apparatus while the X-ray source radiates the X-rays.

The controller may be configured to determine whether the X-ray source irradiates the X-rays, when the X-ray source irradiates the X-rays, to compare a magnitude of the vibration data obtained by the sensing device with a predetermined reference value, to determine the damping coefficient of the damper such that the magnitude of the obtained vibration data is equal to or less than the reference value, and to apply the current to the damper to operate the damper according to the determined damping coefficient.

The sensing device may include at least one of an acceleration sensor, a vibration sensor, a position sensor, and a displacement sensor.

The gantry may include a rotating frame configured to rotate according to a predetermined rotation axis. The sensing device may be provided in plurality in the rotating frame of the gantry. The plurality of sensing devices may be spaced apart from each other according to a predetermined distance on the rotating frame.

The moving device may include a plurality of wheels configured to move the medical imaging apparatus provided with the gantry in a direction of scanning the object. The sensing device may be provided in plurality in correspondence with each of the plurality of wheels.

The image capturing protocol may include at least one of a capturing part of the object, a distance that the gantry moves to scan the object, a moving speed of the gantry, and a rotation speed of the gantry.

In accordance with another aspect of the disclosure, a method of controlling a medical imaging apparatus including a gantry provided to rotate, an X-ray source provided inside the gantry and configured to irradiate X-rays, a moving device configured to move the medical imaging apparatus provided with the gantry, and a damper configured to attenuate vibration generated by movement of the gantry or movement of the medical imaging apparatus, the method includes receiving, by an inputter, an image capturing protocol for an object; determining, by a controller, a damping coefficient of the damper for attenuating the vibration generated by the movement of the gantry or the movement of the medical imaging apparatus corresponding to the input image capturing protocol; and applying, by the controller, a current to the damper to operate the damper according to the determined damping coefficient.

The method may further include storing, by a storage, data about the damping coefficient predetermined according to the image capturing protocol. The predetermined damping coefficient may include at least one of a damping coefficient for attenuating the vibration generated by rotation of the gantry and a damping coefficient for attenuating the vibration generated as the gantry moves in a direction of scanning the object.

The controlling of the operation of the damper may include applying the current to the damper to change the amount of fluid flowing in the damper or to change the viscosity of the fluid for operating the damper according to the determined damping coefficient.

The changing of the amount of fluid flowing inside the damper may include controlling the opening and closing of a piston channel provided in the damper by applying the current to the damper to change the amount of fluid flowing in the damper.

The changing of the viscosity of the fluid may include applying the current to an electromagnet provided inside the damper to form a magnetic field inside the damper to change the viscosity of the fluid.

The method may further include obtaining, by a sensing device, vibration data generated by the movement of the gantry or the movement of the medical imaging apparatus while the X-ray source radiates the X-rays.

The method may further include determining, by the controller, whether the X-ray source irradiates the X-rays; when the X-ray source irradiates the X-rays, comparing, by the controller, a magnitude of the vibration data obtained by the sensing device with a predetermined reference value; determining, by the controller, the damping coefficient of the damper such that the magnitude of the obtained vibration data is equal to or less than the reference value; and applying, by the controller, the current to the damper to operate the damper according to the determined damping coefficient.

Additional aspects of the disclosure will be set forth in part in the description which follows and, in part, will be obvious from the description, or may be learned by practice of the disclosure.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a schematic view of a mobile computed tomography (CT) apparatus according to an embodiment of the disclosure;

FIG. 2 is a view for describing each configuration of the mobile CT apparatus according to an embodiment of the disclosure;

FIGS. 3 to 5 are views for describing in detail a moving device of the mobile CT apparatus according to an embodiment of the disclosure;

FIGS. 6 and 7 are views illustrating that a vibration occurs from movement of a CT apparatus according to an embodiment of the disclosure;

FIGS. 8 and 9 are views illustrating an installation position of a damper and a sensing device according to an embodiment of the disclosure;

FIG. 10 is a view illustrating a configuration of the damper according to an embodiment of the disclosure;

FIG. 11 is a view illustrating an operation principle of the damper according to an embodiment of the disclosure;

FIGS. 12 and 13 are flowcharts illustrating a method of controlling a medical imaging apparatus according to an embodiment of the disclosure; and

FIGS. 14 and 15 are views illustrating controlling a damper according to each operation of the medical imaging apparatus according to an embodiment of the disclosure.

### DETAILED DESCRIPTION

The disclosure and methods of accomplishing the same may be understood more readily by reference to the following detailed description of embodiments and the accompanying drawings. However, the disclosure may be embodied in many different forms, and should not be construed as being limited to the embodiments set forth herein. Rather, these embodiments are provided so that this disclosure will be through and complete and will fully convey the concept of the invention disclosure to those skilled in the art, and the disclosure will only be defined by the appended claims. Throughout the specification, like reference numerals refer to like elements.

The terminology used herein will be described briefly, and the disclosure will be described in detail.

The terminology used herein is defined in consideration of the function of corresponding components used in the disclosure and may be varied according to users, operator's intention, or practices. In addition, an arbitrary defined terminology may be used in a specific case and will be described in detail in a corresponding description paragraph. Therefore, the terminology used herein is for the purpose of describing particular embodiments only and is not intended to be limiting of the disclosure.

When a part “includes” or “comprises” an element, unless there is a particular description contrary thereto, the part can further include other elements, not excluding the other elements. In the following description, terms such as “part”, “module” and “unit” indicate a unit for processing at least one function or operation, wherein the unit and the block may be embodied as software or hardware, such as Field Programmable Gate Array (FPGA), Application Specific Integrated Circuit (ASIC), or embodied by combining hardware and software. However, the term “part” “module” and “unit” are not limited to software or hardware. Further, “part” “module” and “unit” may be constructed to exist in an addressable storage module, or to play one or more processors. “part” “module” and “unit” includes elements (e.g., software elements, object-oriented software elements, class elements and task elements), processors, functions, properties, procedures, sub-routines, segments of a program code, drivers, firmware, a microcode, a circuit, data, a database, data structures, tables, arrays, and variables. The function provided in the components and “part” may be combined into fewer components and “part” or further separated into additional components and the “part”.

In the following detailed description, only certain exemplary embodiments of the disclosure have been shown and described, simply by way of illustration. However, the disclosure may be embodied in many different forms and should not be construed as limited to the embodiments set forth herein. The drawings and description are to be regarded as illustrative in nature and not restrictive.

Throughout the specification, an “image” may refer to multi-dimensional data formed of discrete image elements (e.g., pixels in a two-dimensional (2D) image and voxels in a three-dimensional (3D) image). For example, the image may include a medical image of an object obtained by a computed tomography (CT) imaging apparatus.

Throughout the specification, a “CT image” may refer to an image synthesized using a plurality of X-ray images obtained by photographing an object while a CT imaging apparatus rotates about at least one axis with respect to the object.

Throughout the specification, an “object” may refer to a human, an animal, or a part of a human or animal. For example, the object may include at least one of organs such as liver, heart, womb, brain, breast, and abdomen and blood vessels. Also, the “object” may be a phantom. The phantom refers to a material having a volume very close to a density and effective atomic number of an organism and may include a sphere phantom having characteristics similar to those of a human body.

As used herein, a “user” may refer to a medical professional, such as a doctor, a nurse, a medical laboratory technologist, a medical imaging professional, and a medical equipment technician, and the like, without being limited thereto.

Since a CT system is capable of providing cross-sectional images of an object, the CT apparatus may express an inner structure (e.g., an organ such as a kidney and a lung) of the object without an overlap between discrete portions of the object, compared to conventional X-ray imaging apparatuses.

The CT system may include any imaging apparatuses that obtain tomograms such as a computed tomography (CT) apparatus, an optical coherence tomography (OCT) apparatus, and a positron emission tomography (PET)-CT apparatus.

Hereinafter, the CT system will be described as an example of the CT apparatus.

The CT apparatus may obtain a plurality of image data each with a thickness of 2 mm or less for several tens to several hundreds of times per second and then process the obtained data, thereby providing a relatively accurate cross-sectional image of the object. Although only horizontal cross-sectional images of the object have been obtained according to the related art, this issue has been overcome due to various image construction methods below. Examples of 3D image reconstruction methods are as follows.
Shade Surface Display (SSD): An initial imaging method that displays only voxels having a predetermined Hounsfield Unit (HU) value.
Maximum Intensity Projection (MIP)/Minimum Intensity Projection (MinIP): A 3D imaging method that displays only voxels having the highest or lowest HU value among voxels constructing an image.
Volume Rendering (VR): An imaging method capable of adjusting color and transmittance of voxels on the basis of region of interest that construct an image.
Virtual Endoscopy: A method allowing endoscopic observation in a 3D image reconstructed by VR or SSD methods.
Multi Planar Reformation (MPR): A method of reconstructing an image into a different cross-sectional image. A user may reconstruct an image in a desired direction.
Editing: A method of editing adjacent voxels so as to allow a user to easily observe a region of interest in volume rendering.
Voxel of Interest (VOI): A method of displaying only a selected area in volume rendering.

The CT apparatus according to an embodiment will be described with reference to the accompanying drawings. The CT apparatus may include apparatuses in various shapes.

In addition, the CT apparatus may be an imaging device requiring a scout scan or a pre-shot operation.

It is an aspect of the disclosure to provide a medical imaging apparatus capable of attenuating vibration caused by movement of a gantry or movement of the medical imaging apparatus by controlling a current applied to a damper provided in the medical imaging apparatus, and a method of controlling the medical imaging apparatus.

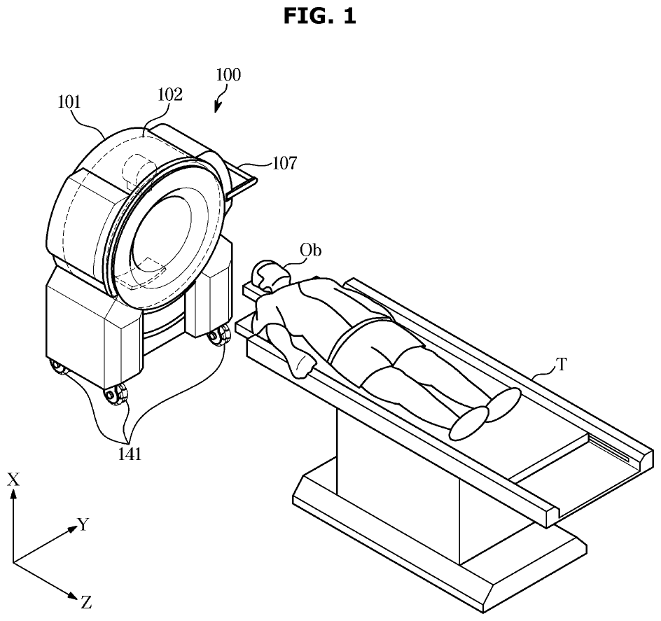

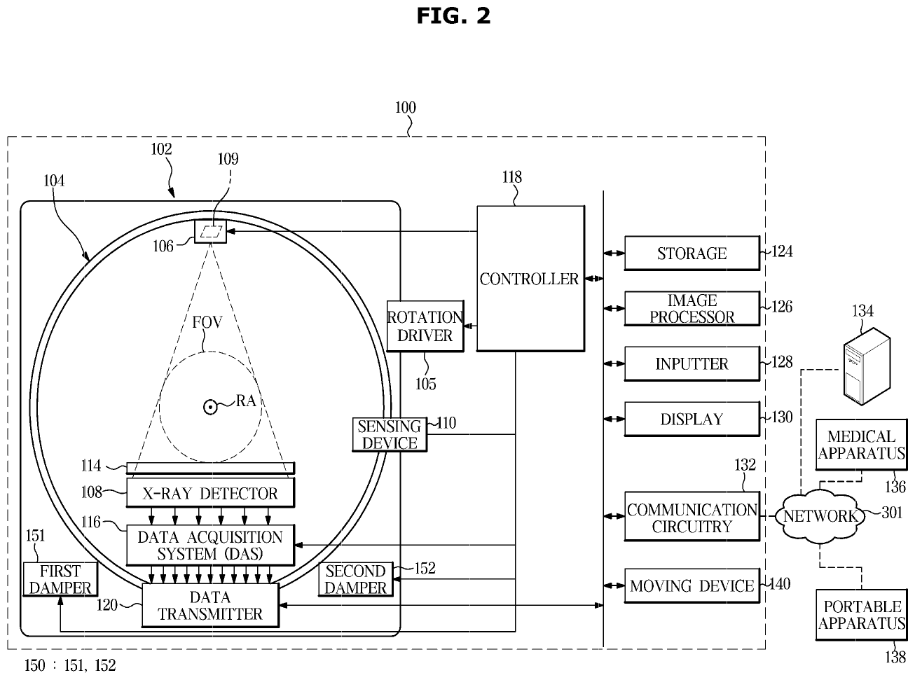

FIG. 1 is a schematic view of a mobile computed tomography (CT) apparatus according to an embodiment of the disclosure, and FIG. 2 is a view for describing each configuration of the mobile CT apparatus according to an embodiment of the disclosure. In order to avoid overlapping description, it describes together below.

Referring to FIG. 1, a mobile CT apparatus 100 may include a main body 101 including a gantry 102, a handle 107, and a wheel 141.

The mobile CT apparatus 100 may move through the wheel 141, and may be moved on the Y-axis or Z-axis by a moving device 140 providing a driving force to the wheel 141 as well as a force provided by the user through the handle 107.

Meanwhile, the mobile CT apparatus 100 may perform tomography while moving in the Z-axis direction based on a table T where an object Ob is located. A detailed description thereof will be described later with reference to other drawings.

Referring to FIG. 2, the gantry 102 may include a rotating frame 104, an X-ray source 106, an X-ray detector 108, a rotation driver 105, a data acquisition system (DAS) 116, and a data transmitter 120.

The gantry 102 may include the rotating frame 104 having a loop shape and rotatable with respect to a predetermined rotation axis (RA). Also, the rotating frame 104 may have a disc shape.

The rotating frame 104 may have the X-ray source 106 and the X-ray detector 108 that face each other to have predetermined fields of view (FOVs). The rotating frame 104 may further include an anti-scatter grid 114. The anti-scatter grid 114 may be disposed between the X-ray source 106 and the X-ray detector 108.

As the rotating frame 104 rotates, the gantry 102 may rotate about 180 to 360 degrees around a bore. As the gantry 102 rotates, the X-ray source 106 and the X-ray detector 108 may also rotate.

X-ray radiation that arrives at a detector (or photosensitive film) includes not only attenuated primary radiation that forms a valuable image but also scattered radiation that deteriorates the quality of image. In order to transmit most of the primary radiation and attenuate the scattered radiation, the anti-scatter grid 114 may be disposed between a patient and the detector (or photosensitive film).

For example, the anti-scatter grid 114 may be formed by alternately stacking strips of lead foil and an interspace material such as a non-porous solid polymer material or a fiber composite material. However, the structure of the anti-scatter grid 114 is not limited thereto.

The rotating frame 104 may receive a driving signal from the rotation driver 105 and rotate the X-ray source 106 and the X-ray detector 108 at a predetermined rotation speed. The rotating frame 104 may receive the driving signal and power from the rotation driver 105 while the rotating frame 104 contacts the sensing device 110 via a slip ring (not shown). Also, the rotating frame 104 may receive the driving signal and power from the rotation driver 105 via a wireless communication network.

The X-ray source 106 may generate and emit X-rays by receiving a voltage or current from a power distribution unit (PDU) (not shown) via a slip ring (not shown) and then a high voltage generator (not shown). When the high voltage generator applies a predetermined voltage (hereinafter, referred to as tube voltage) to the X-ray source 106, the X-ray source 106 may generate X-rays having a plurality of energy spectra that correspond to the tube voltage.

X-rays generated by the X-ray source 106 may be emitted in a predetermined form or into a predetermined region by a collimator 109.

The X-ray detector 108 may be positioned to face the X-ray source 106. The X-ray detector 108 may include a plurality of X-ray detecting elements. Although each of the plurality of X-ray detecting elements may establish one channel, the embodiments of the disclosure are not limited thereto.

The X-ray detector 108 may detect X-rays generated by the X-ray source 106 and received through the object Ob and generate electrical signals corresponding to the intensity of the detected X-rays.

The X-ray detector 108 may include an indirect-type X-ray detector configured to detect radiation after converting the radiation into light and a direct-type X-ray detector configured to detect radiation after directly converting the radiation into electric charges. A scintillator may be used as the indirect-type X-ray detector. In addition, a photon counting detector may be used as the direct-type X-ray detector.

The DAS 116 may be connected to the X-ray detector 108. The electrical signals generated by the X-ray detector 108 may be collected by the DAS 116. The electrical signals generated by the X-ray detector 108 may be collected by the DAS 116 in a wired or wireless manner.

In addition, the electrical signals generated by the X-ray detector 108 may be provided to an analog/digital converter (not shown) via an amplifier (not shown).

Only some of a plurality of pieces of data collected from the X-ray detector 108 may be provided to an image processor 126 according to a slice thickness or the number of slices. Also, the image processor 126 may select only some of the plurality of pieces of data.

Such digital signals may be provided to the image processor 126 via the data transmitter 120. The digital signals may be transmitted to the image processor 126 in a wired or wireless manner via the data transmitter 120.

A sensing device 110 may collect various data inside and outside the mobile CT apparatus 100.

In detail, the sensing device 110 may obtain vibrations generated by the rotation of the gantry 102 and vibration data generated while the mobile CT apparatus 100 moves for tomography.

As described above, instead of fixing the table T, the mobile CT apparatus 100 may capture the object Ob while the mobile CT apparatus 100 moves in the Z-axis direction. Accordingly, a space in which the mobile CT apparatus 100 performs tomography may be very diverse, and the gantry 102 moving by the moving device 140 may be shaken.

The sensing device 110 may be provided at a predetermined position of the rotating frame 104 or may be provided on a wheel for moving the gantry 102 to detect vibrations resulting from the rotation or movement of the gantry 102 and obtain vibration data.

The vibration data measured by the sensing device 110 may include various data such as a moving value measured while the main body 101 moves, a rotation angle measured by the shifting direction of the main body 101 due to an uneven floor or obstacle, or vibration value that may occur while the gantry 102 is rotated by the rotation driver 105.

The sensing device 110 may include various hardware sensors capable of collecting position data, such as an acceleration sensor, a vibration sensor, a position sensor, and a displacement sensor. Each sensor provided in hardware may be provided in the main body 101 in plural instead of singular.

In detail, a plurality of sensing devices 110 may be provided in the rotating frame 104, and the plurality of sensing devices 110 may be spaced apart from the rotating frame 104 by a predetermined distance. In addition, the sensing device 110 may be provided in plurality in correspondence with each of the plurality of wheels for moving the gantry. A detailed description thereof will be described later with reference to other drawings.

Meanwhile, the vibration data obtained by the sensing device 110 may be transmitted to a controller 118, and the controller 118 may compare the vibration data obtained by the sensing device 110 with a predetermined reference value.

The controller 118 may be a component for controlling the overall operation of the mobile CT apparatus 100. The controller 118 may be implemented using a memory (not shown) that stores data on algorithms to control the operation of each of the modules included in FIG. 2 or data on programs to run the algorithms and a processor (not shown) that performs the aforementioned operation by using data stored in the memory. In this case, the memory and the processor may be implemented as separate chips. Alternatively, the memory and the processor may be implemented as a single chip.

As described below, the controller 118 may control the operation of the dampers 150 (151 and 152) to attenuate the vibration generated by the rotation and movement of the gantry 102 according to an image capturing protocol input for capturing the object Ob by the mobile CT apparatus 100.

In addition, the controller 118 may determine whether X-rays are irradiated to the object Ob from the X-ray source 106, and while the X-ray source 106 irradiates the X-rays, the controller 118 may control the operation of a damper 150 by comparing the magnitude of the vibration data obtained by the sensing device 110 with a reference value previously stored in a storage 124.

The storage 124 stores data obtained from the DAS 116 and data measured by the sensing device 110.

In addition, the storage 124 may store data necessary for controlling the damper 150 to be described later. In detail, data about a predetermined damping coefficient may be stored according to the image capturing protocol input for capturing the object. The damping coefficient may include a damping coefficient for attenuating the vibration generated by the rotation of the gantry 102 and a damping coefficient for attenuating the vibration generated as the gantry 102 moves in the direction in which the object is scanned.

In addition, when the X-ray source 106 irradiates X-rays, the storage 124 may store the predetermined vibration value that is a reference to the magnitude of the vibration data obtained by the sensing device 110.

The controller 118 may control the damper 150 to attenuate the vibration generated by the movement of the gantry 106 based on the damping coefficient of the damper 150 stored in the storage 124 or the magnitude value of the vibration data.

In addition, the storage 124 may store various data such as image data that has been processed image processing and tomographic images generated through image data.

The storage 124 may include at least one type storage medium selected from a flash memory type storage medium, a hard disk type storage medium, a multimedia card micro type storage medium, a card type memory (e.g., SD card and XD memory), Random Access Memory (RAM), Static Random Access Memory (SRAM), Read-Only Memory (ROM), Electrically Erasable Programmable Read-Only Memory (EEPROM), Programmable Read-Only Memory (PROM), a magnetic memory, a magnetic disc, and an optical disc.

The image processor 126 may receive data (e.g., pure data before processing), which is obtained from the DAS 116 through the data transmitter 120, and may perform pre-processing and generate a tomogram using the image data generated through the pre-processing. Particularly, the pre-processing may include a process of correcting sensitivity irregularity between channels, a process of correcting a signal loss due to a rapid decrease of a signal strength or due to an X-ray absorbing material such as metal, or the like.

The image data pre-processed by the image processor 126 may be referred to as raw data or projection data. The projection data may be a group of data values corresponding to the intensities of the X-rays that pass through the object. For descriptive convenience, a group of a plurality of pieces of projection data that are simultaneously obtained from all channels in the same imaging angle is referred to as a projection data set.

The image processor 126 may generate a primary cross-sectional image using the obtained projection data set and generate a secondary cross-sectional image of the object by reconstructing the primary cross-sectional image. The secondary cross-sectional image may be a 3D image. In other words, the image processor 126 may generate a 3D X-ray tomogram of the object by cone beam reconstruction based on the obtained projection data set.

Meanwhile, the image processor 126 may be implemented using a memory that stores data on algorithms to convert digital data into an image or perform image processing or data on programs to run the algorithms and a graphics processing unit (GPU) that performs the aforementioned operation by using the data stored in the memory. In this case, the memory and the GPU may be implemented as separate chips. Alternatively, the memory and the GPU may be implemented as a single chip.

Various user input commands for X-ray tomography conditions, image processing conditions, and the like may be received through an inputter 128. For example, the X-ray tomography conditions may include a plurality of tube voltages, energy value settings with respect to a plurality of X-rays required for general tomography, selection of the image capturing protocol, selection of an image reconstruction method, setting of a field of view (FOV) area, the number of slices, a slice thickness, parameter setting with respect to post-processing of image, and the like as well as a start of operation of the mobile CT apparatus 100 and moving conditions of the moving device 140.

In addition, the inputter 128 may receive the image capturing protocol for the mobile CT apparatus 100 to capture the object. The image capturing protocol may vary depending on a type of object to be captured and a capturing part of the object. In addition, the image capturing protocol may include information such as a distance that the gantry 102 moves while scanning the object, an object scan moving speed of the gantry 102, and a rotation speed of the gantry 102 to capture the object.

That is, the mobile CT apparatus 100 may change a capturing form according to an input image capturing protocol, and capturing may be started and proceeded.

The inputter 128 may include a device for receiving a predetermined input from the outside. For example, inputter 128 may include hardware devices such as various buttons or switches, pedals, keyboards, mice, track-balls, various levers, handles or sticks, and the like.

A display 130 may display an X-ray tomographic image reconstructed by the image processor 126 and various interfaces. For example, the display 130 may display a monochrome radiation image generated by the image processor 126.

Meanwhile, when the display 130 is implemented as a touch screen panel (TSP), the display 130 may form a mutual layer structure with the inputter 128. In this case, the inputter 128 may include a graphical user interface (GUI) such as a touch pad, that is, a device that is software.

A communication circuitry 132 may perform communication with an external device, an external medical apparatus, or the like through a server 134, or the like.

The communication circuitry 132 may be connected to a network 301 in a wired or wireless manner and perform communication with the server 134, an external medical apparatus 136, or a portable apparatus 138 that is external. The communication circuitry 132 may exchange data with a hospital server or other medical apparatuses in a hospital via a picture archiving and communication system (PACS).

In addition, the communication circuitry 132 may perform data communication with the portable apparatus 138 or the like according to a Digital Imaging and Communications in Medicine (DICOM) standard.

The communication circuitry 132 may transmit and receive data related to diagnosis of the object via the network 301. In addition, the communication circuitry 132 may transmit and receive a medical image obtained by the medical apparatus 136 such as an MRI apparatus and an X-ray apparatus.

Furthermore, the communication circuitry 132 may receive a diagnosis history or a medical treatment schedule of a patient from the server 134 and use the diagnosis history or the medical treatment schedule in a clinical diagnosis of the patient. In addition, the communication circuitry 132 may perform data communication with not only the server 134 or the medical apparatus 136 in a hospital but also with the portable apparatus 138 of the user or patient.

Also, the communication circuitry 132 may transmit information on malfunction, a quality management status, or the like to a system manager or a service manager through the network and may receive feedback corresponding to the information.

The moving device 140 may move the main body 101 of the mobile CT apparatus 100.

That is, the moving device 140 may move the position of the gantry 102 so that the gantry 102 of the mobile CT apparatus 100 can scan the object.

In more particular, the moving device 140 may include a plurality of motors (not shown) that provide the driving force to the plurality of wheels 141. The mobile CT apparatus 100 may be divided into a first wheel 141 providing a rotational force and a second wheel 142 used in tomography so that a user can freely move the main body 101 through the handle 107.

That is, the mobile CT apparatus 100 may move to the object Ob located on the table T using the first wheel 141, and may then start capturing using the second wheel. To this end, the first wheel 141 may be provided in a chain shape to support the main body 101 and to have a large radius of rotation, and the second wheel may be provided to be moved for precise capturing in mm units.

The object to which the moving device 140 moves may be the main body 101 in which the structure of the mobile CT apparatus 100 is provided. However, hereinafter, the moving device 140 will be described as moving the gantry 102, for convenience of description.

The damper 150 may attenuate the vibration generated by the rotation or movement of the gantry 102.

The damper 150 may be applied with a current under the control of the controller 118, and the operation of the damper 150 may be controlled according to the predetermined damping coefficient to attenuate the vibration.

The damper 150 may be provided in plural in the mobile CT apparatus 100, and may include a first damper 151 and a second damper 152 as illustrated in FIG. 2. A description of the damper 150 will be described later in detail with reference to the other drawings.

Components may be added or deleted corresponding to performance of the components of the mobile CT apparatus 100 illustrated in FIGS. 1 and 2. In addition, it will be readily understood by those skilled in the art that mutual positions of the components may be changed to correspond to performance or structure of a system. Some of the elements illustrated in FIGS. 1 and 2 may be a software and/or hardware components such as a Field Programmable Gate Array (FPGA) or an Application Specific Integrated Circuit (ASIC).

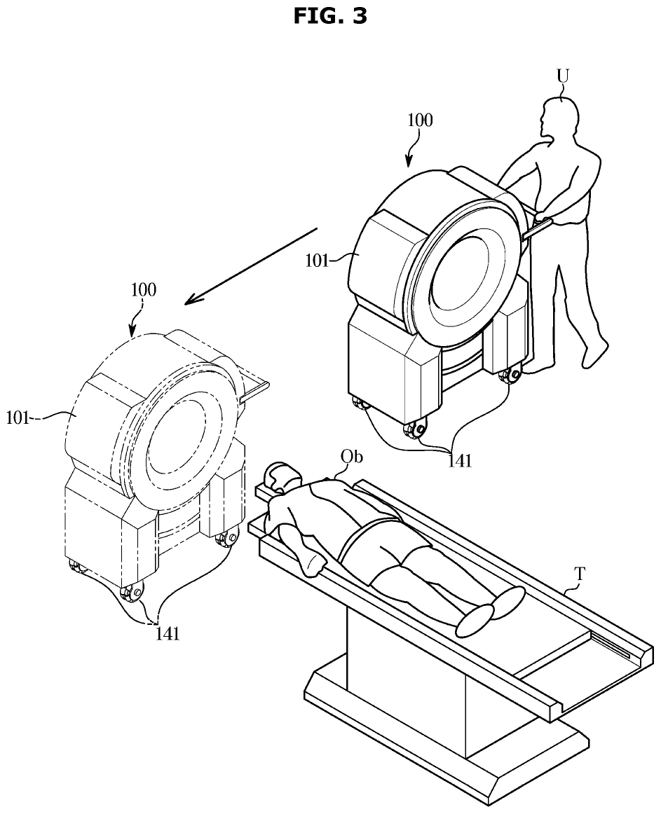

FIGS. 3 to 5 are views for describing in detail a moving device of the mobile CT apparatus according to an embodiment of the disclosure. In order to avoid overlapping description, it describes together below.

Referring to FIG. 3, a user U may move the mobile CT apparatus 100 near the object Ob through the first wheel 141. In detail, the user U may move the mobile CT apparatus 100 through the first wheel 141 to a position corresponding to an anatomical position at which the object Ob is to be captured.

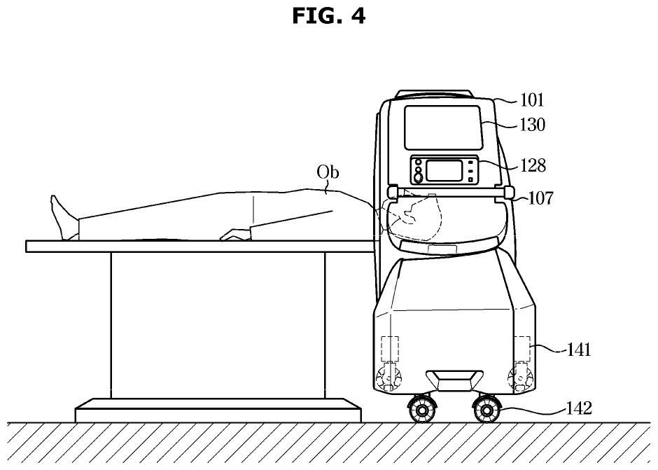

For example, the user U may move the mobile CT apparatus 100 to a position of FIG. 4 in order to capture a head of the object Ob. When it is determined that the mobile CT apparatus 100 is moved to a position suitable for capturing, the user U may replace the first wheel 141 with the second wheel 142.

The mobile CT apparatus 100 may replace the first wheel 141 and the second wheel 142 through the inputter 128. After the second wheel 142 is completely protruded from the inside the main body 101, the first wheel 141 may be inserted into the main body 101.

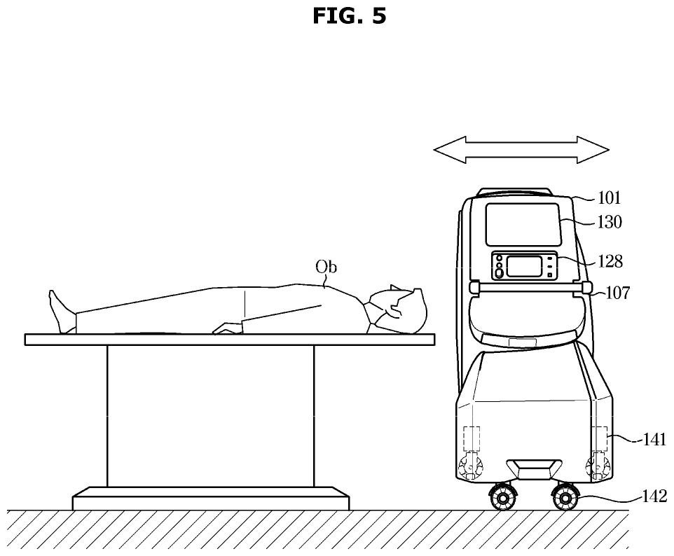

Referring to FIG. 5, the inputter 128 may receive an input command regarding the start of capturing from the user U. The mobile CT apparatus 100 may rotate the gantry 102 through the rotation driver 105. In addition, the mobile CT apparatus 100 may move straight through the second wheel 142 in the Z-axis direction, particularly, in a direction approaching or away from the object Ob.

When the mobile CT apparatus 100 moves while rotating the gantry 102, the sensing device 110 may detect the position data, and the controller 118 may perform the image processing using the position data detected in the image processing process.

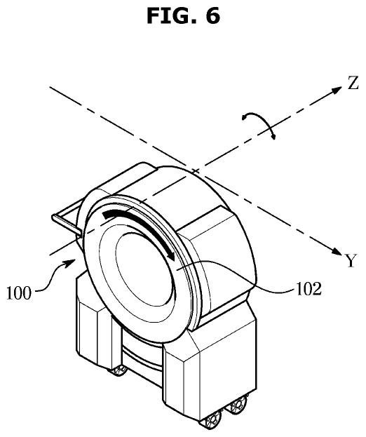

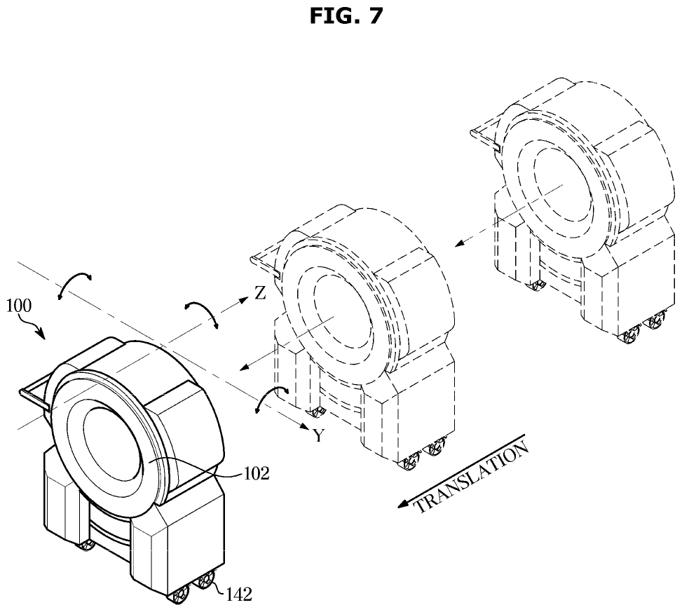

FIGS. 6 and 7 are views illustrating that a vibration occurs from movement of a CT apparatus according to an embodiment of the disclosure.

Referring to FIG. 6, when the gantry 102 rotates, the vibration occurs in the left and right directions with respect to the Z-axis.

As will be described later, the gantry 102 may be accelerated to a predetermined speed to reach the predetermined speed and then rotate at the constant speed. When the gantry 102 is accelerated in a clockwise direction as illustrated in FIG. 6, a tipping phenomenon in which the gantry 102 is accelerated to the right side of the gantry 102, that is, in the clockwise direction of rotation, occurs, and thus, a shake occurs based on the Z-axis, which is the rotation axis of the gantry 102.

In addition, when the mobile CT apparatus 100 moves in the Z-axis direction by the moving device 140 as illustrated in FIG. 7, the front and back shake occurs.

That is, when the gantry 102 moves along the Z-axis to scan the object, a shaking occurs in a moving direction and an opposite direction.

When the gantry 102 is moved to scan the object, the gantry 102 moves in the Z-axis direction while the gantry 102 is rotating, and thus the shaking occurs based on the Z-axis according to the rotation direction of the gantry 102. At the same time, the shaking occurs based on the Y-axis according to the movement of the gantry 102.

In addition, when a ground is uneven while the gantry 102 moves, the shaking may be increased, and when a moving speed of the gantry 102 is high or a moving distance is long, the shaking of the gantry 102 may be increased.

That is, the vibration generated according to the movement of the gantry 102 may vary in a generation position of the vibration, the vibration direction, and the magnitude of the vibration depending on the movement of the gantry 102 operating according to the image capturing protocol for capturing the object.

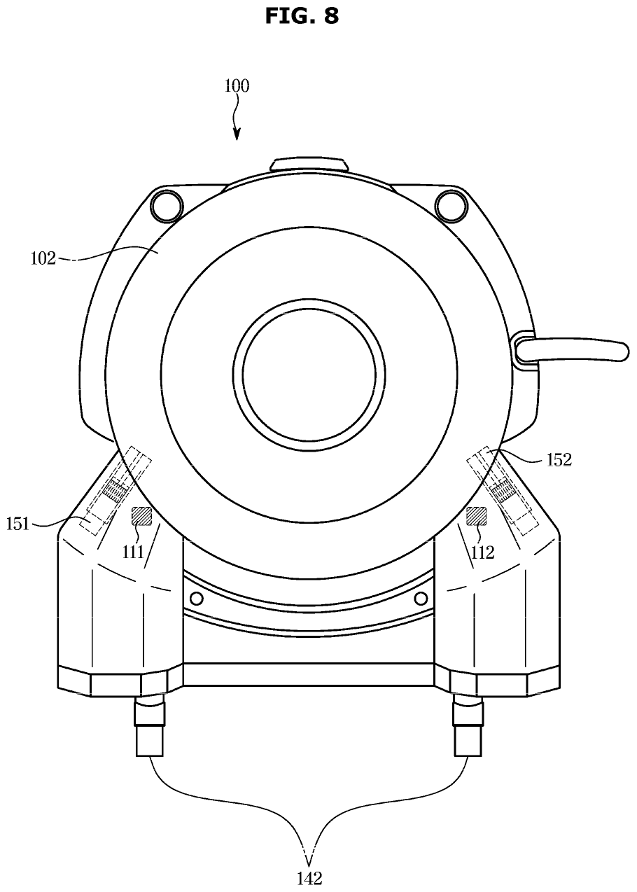

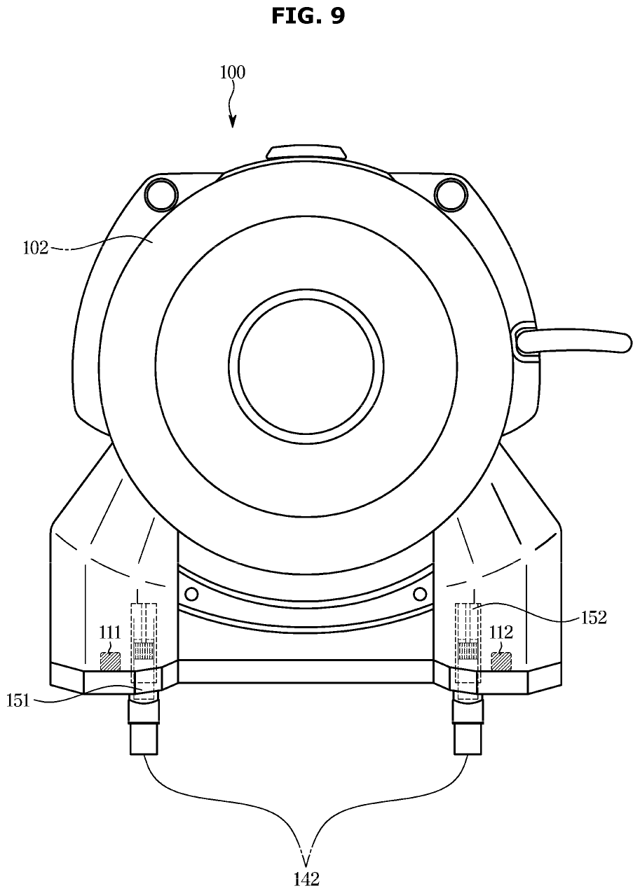

FIGS. 8 and 9 are views illustrating an installation position of a damper and a sensing device according to an embodiment of the disclosure.

The damper 150 may be configured to attenuate the vibration generated by the movement of the gantry 102, and a plurality of dampers 150 may be provided at positions adjacent to the gantry 102.

Referring to FIG. 8, the damper 150 may include the first damper 151 and the second damper 152. The first damper 151 may be provided on the left side of the rotating frame 104, and the second damper 152 may be provided on the right side of the rotating frame 104.

That is, on the rotating frame 104, the first damper 151 and the second damper 152 may be spaced apart from each other according to a predetermined distance.

Since the first damper 151 and the second damper 152 are configured to attenuate the vibration generated by the rotation of the gantry 102 and the movement of the gantry 102, the first damper 151 and the second damper 152 may be installed on the rotating frame 104 of the gantry 102 to reduce the vibration generated by the gantry 102 under the control of the controller 118.

For example, as described above with reference to FIG. 6, when the phenomena occurs in which the gantry 102 is tilted to the right side of the gantry 102 and the vibration occurs because the gantry 102 accelerates and rotates the clockwise direction, the controller 118 may reduce the vibration of the gantry 102 by controlling damping characteristics by applying the current to the second damper 152 provided on the right side of the rotating frame 104.

As described above, the damper 150 may be provided as the first damper 151 and the second damper 152 on the rotating frame 104 of the gantry 102, but the installation position and the number of the damper 150 is not limited. Any position may be used as long as it can be controlled to reduce the vibration generated by the movement of the gantry 102.

Similarly, the sensing device 110 is configured to obtain the vibration data generated by the movement of the gantry 102 while the X-rays are radiated from the X-ray source 106, and a plurality of sensing devices 110 may be provided at positions adjacent to the gantry 102.

As illustrated in FIG. 8, the sensing device 110 may include a first sensing device 111 and a second sensing device 112. The first sensing device 111 may be provided on the left side of the rotating frame 104, and the second sensing device 112 may be provided on the right side of the rotating frame 104.

That is, on the rotating frame 104, the first sensing device 111 and the second sensing device 112 may be spaced apart from each other by the predetermined distance.

The first sensing device 111 and the second sensing device 112 may be installed on the rotating frame 104, and the first sensing device 111 and the second sensing device 112 may obtain, in real time, the vibration data generated from the rotation of the gantry 102 and the movement of the gantry 102 while the X-rays are irradiated from the X-ray source 106, and then transmit the obtained vibration data to the controller 118.

As described above, the sensing device 110 may be provided as the first sensing device 111 and the second sensing device 112 on the rotating frame 104 of the gantry 102, but the installation position and the number of the sensing device are limited. Any position may be used as long as it can obtain the vibration generated from the movement of the gantry 102 in real time.

Alternatively, the damper 150 may be provided on the second wheel 142 for moving the gantry 102. That is, as illustrated in FIG. 9, the first damper 151 and the second damper 152 may be provided in each of the plurality of second wheels 142.

The first damper 151 and the second damper 152 may be provided on two second wheels 142 to reduce the vibration generated as the gantry 102 rotates by the second wheel 142 in a scan direction of the object.

When the number of second wheels 142 is different, the total number of dampers 150 provided in each of the second wheels 142 may vary.

When the gantry 102 moves, the direction or position of the shaking occurring in the gantry 102 may vary according to the moving direction or the moving speed. Therefore, the controller 118 may control the damping characteristics of each of the first damper 151 and the second damper 152 provided in each of the plurality of second wheels 142 to reduce the vibration generated differently according to the movement type of the gantry 102.

As described above, the damper 150 may be provided as the first damper 151 and the second damper 152 on each of the plurality of second wheels 142 moving the gantry 102 to scan the object. The installation position and the number of the damper are not limited, and any position may be used as long as it can be controlled to reduce the vibration generated by the movement of the gantry 102.

Similarly, the first sensing device 111 and the second sensing device 112 may be provided in each of the plurality of second wheels 142 to obtain the vibration data generated by the movement of the gantry 102 while the X-rays are irradiated from the X-ray source 106.

As described above, the sensing device 110 may be provided as the first sensing device 111 and the second sensing device 112 on each of the plurality of second wheels 142 moving the gantry 102 to scan the object. The installation position and the number of the sensing device are not limited, and any position may be used as long as it can obtain the vibration generated from the movement of the gantry 102 in real time.

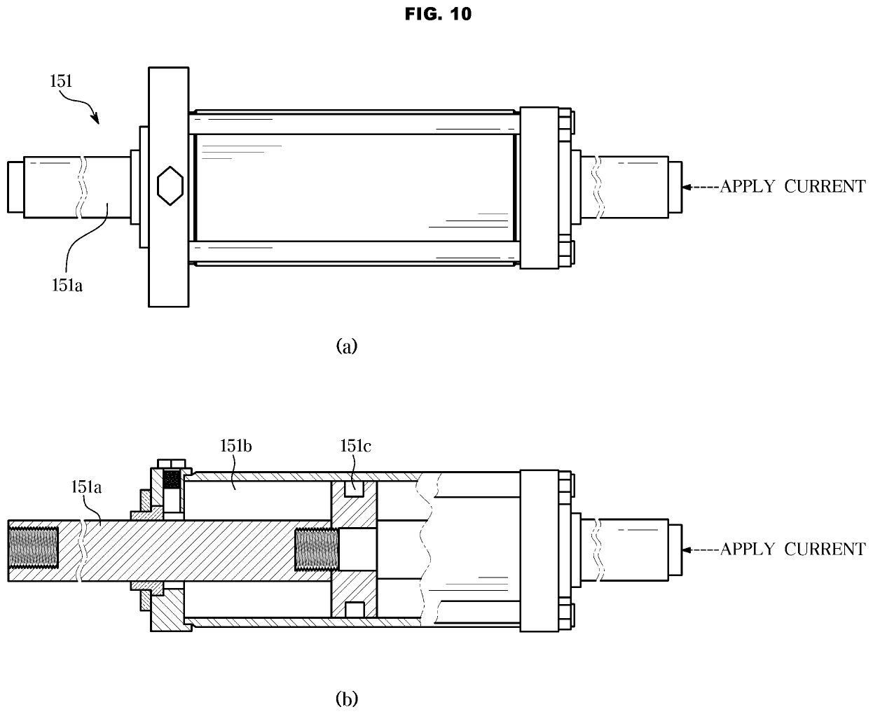

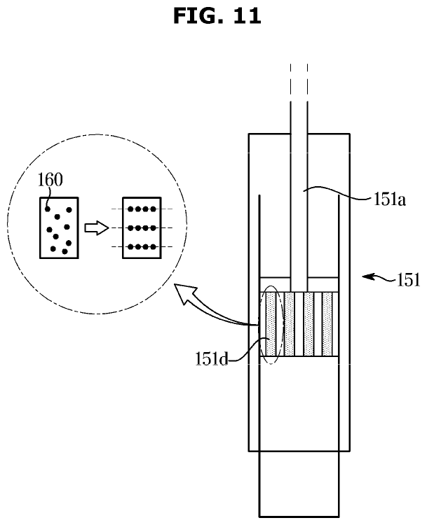

FIG. 10 is a view illustrating a configuration of the damper according to an embodiment of the disclosure, and FIG. 11 is a view illustrating an operation principle of the damper according to an embodiment of the disclosure.

Referring to FIG. 10, the damper 150 may be implemented as a dynamic damper that is commonly used.

The damper 150 may include a piston 151a and an electromagnet 151c that forms a magnetic field according to the application of a current therein. A fluid 151b may be included in the damper 150. The fluid 151b may generally be damping oil for adjusting a damping force of the damper 150.

In addition, the piston 151a may include a piston channel 151d serving as a passage through which the fluid 151b can flow, and the fluid 151b may include metal particles 160.

The controller 118 may apply the current to the damper 150, and the damper 150 may operate according to the predetermined damping coefficient by changing the amount of fluid 151b flowing inside the damper 150 or changing the viscosity of the fluid 151b according to the application of the current.

That is, by controlling the damper 150 to operate according to the predetermined damping coefficient under the control of the controller 118, the damping force of the damper 150 may be adjusted to attenuate the vibration caused by shaking generated in the device in which the damper 150 is installed.

Referring to FIG. 11, the controller 118 may apply the current to the damper 150 and the magnetic field may be formed in the piston channel 151d by the electromagnet 151c according to the applied current.

By the magnetic field formed in the piston channel 151d, the metal particles 160 included in the fluid flowing through the piston channel 151d may be aligned in the direction of the magnetic field. When the metal particles 160 are aligned, the viscosity of the fluid 151b flowing through the piston channel 151d may be increased.

Therefore, when the current is applied to the damper 150, the damper 150 may be firmly operated by increasing the viscosity of the fluid 151b according to the alignment of the metal particles 160. That is, the controller 118 may apply the current to the damper 150 to control the damper 150 to operate with the damping coefficient that can reduce the shaking of the device in which the damper 150 is installed.

On the other hand, when no current is applied to the damper 150, the viscosity of the fluid 151b is lowered, and thus the damper 150 may operate smoothly. That is, the controller 118 may block the current applied to the damper 150 to control the damper 150 to operate with the damping coefficient that can accommodate the shaking of the device in which the damper 150 is installed.

Accordingly, the controller 118 may control the current applied to the damper 150 so that the damper 150 operates according to the predetermined damping coefficient to reduce or to accommodate the shaking of the device in which the damper 150 is installed.

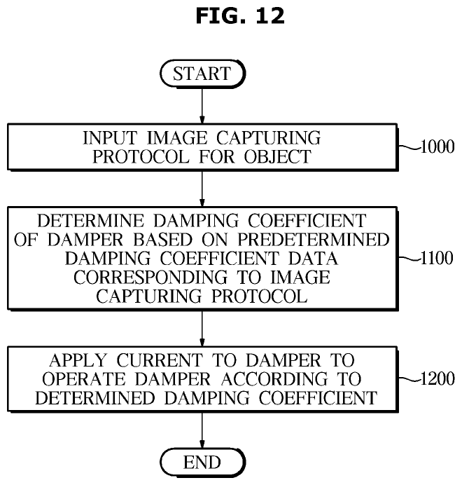

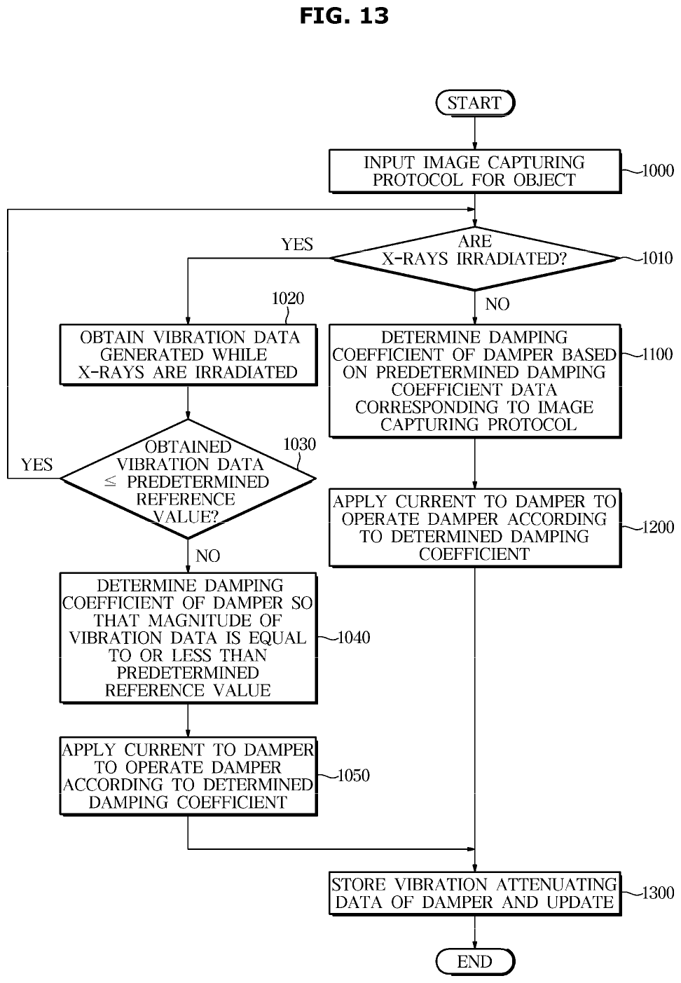

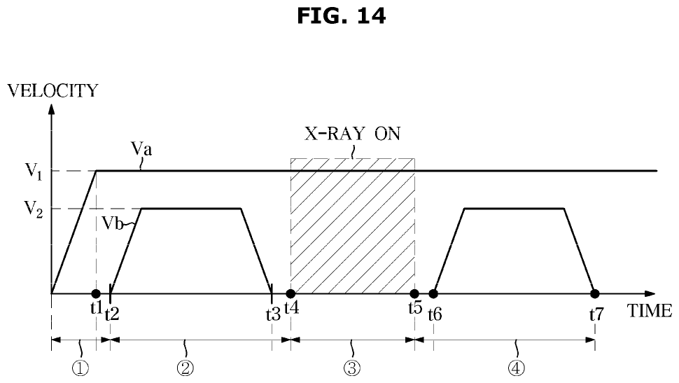

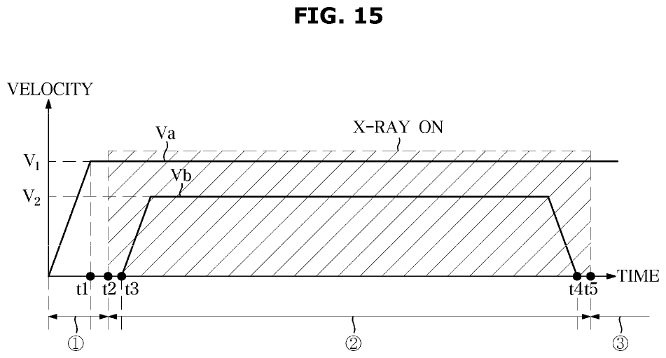

FIGS. 12 and 13 are flowcharts illustrating a method of controlling a medical imaging apparatus according to an embodiment of the disclosure, and FIGS. 14 and 15 are views illustrating controlling a damper according to each operation of the medical imaging apparatus according to an embodiment of the disclosure.

FIG. 12 is a flowchart illustrating a method of controlling a medical imaging apparatus according to an embodiment, and FIG. 13 is a view illustrating the flowchart of FIG. 12 in more detail at each step.

Referring to FIGS. 12 and 13, the user may input the image capturing protocol for the mobile CT apparatus 100 to capture the object through the inputter 128 (1000).

The input image capturing protocol may vary depending on the type of the object to be captured and the capturing part of the object. In addition, the image capturing protocol may include the information such as the distance that the gantry 102 moves while scanning the object, the object scan moving speed of the gantry 102, and the rotation speed of the gantry 102 to capture the object.

The mobile CT apparatus 100 may change the capturing form according to the input image capturing protocol, and the capturing may be started and proceeded. The vibration generated according to the movement of the gantry 102 may vary in the generation position of the vibration, the vibration direction, and the magnitude of the vibration depending on the movement of the gantry 102 operating according to the image capturing protocol for capturing the object.

Therefore, the controller 118 may control the damper 150 to operate according to the predetermined damping coefficient. The storage 124 may store the data necessary for controlling the damper 150.

In detail, data about the predetermined damping coefficient may be stored according to the image capturing protocol input for capturing the object. The damping coefficient may include the damping coefficient for attenuating the vibration generated by the rotation of the gantry 102 and the damping coefficient for attenuating the vibration generated as the object moves in the direction in which the object is scanned.

In addition, when the X-ray source 106 irradiates X-rays, the storage 124 may store the predetermined vibration value that is the reference to the magnitude of the vibration data obtained by the sensing device 110.

The controller 118 may control the damper 150 to attenuate the vibration generated by the movement of the gantry 106 based on the damping coefficient of the damper 150 stored in the storage 124 or the magnitude value of the vibration data.

For example, when the gantry 102 operates according to the input image capturing protocol, the controller 118 may control the damper 150 to reduce the shaking of the gantry 102 by operating with a relatively hard damping force according to the predetermined damping coefficient. On the other hand, the controller 118 may control the damper 150 to accommodate the shaking of the gantry 102 by operating with a relatively soft damping force according to the predetermined damping coefficient.

The controller 118 may first determine whether the X-rays are irradiated to the object from the X-ray source 106 provided in the gantry 102 (1010).

As will be described later, this is because the damper 150 is not controlled according to the predetermined damping coefficient while the X-rays are irradiated to the object, but the operation of the damper 150 may be controller according to the magnitude of the vibration data obtained in real time by the sensing device 110.

When the controller 118 determines that the X-rays are not being irradiated, the controller 118 may determine the damping coefficient of the damper 150 based on the predetermined damping coefficient data corresponding to the input image capturing protocol (1100), and may apply the current to the damper 150 to operate the damper 150 according to the determined damping coefficient (1200).

The graph disclosed in FIG. 14 includes a graph of a rotation speed va of the gantry 102 and a moving speed vb that the gantry 102 moves to scan the object.

Referring to FIG. 14, when the CT of the object is started according to the image capturing protocol input from the user, the gantry 102 starts to rotate and is accelerated to a predetermined speed v1 to reach the predetermined speed v1, and proceeds to the constant speed rotation from a time t1 to v1. That is, the gantry 102 may rotate at the speed of v1 until the capturing of the object is finished.

In a section \textcircled{1} in which the gantry 102 rotates in the clockwise direction and the rotation speed is accelerated, the tipping phenomenon occurs in the right direction of the gantry 102, and thus the shake occurs based on the Z-axis, which is the rotation axis of the gantry 102.

The controller 118 may determine the damping coefficient for attenuating the vibration in an acceleration section of the gantry 102 based on the damping coefficient data of the damper 150 stored in the storage 124 in advance for the section \textcircled{1}, and may apply the current to the damper 150 to operate the damper 150 according to the determined damping coefficient.

Particularly, the controller 118 may reduce the vibration of the gantry 102 by controlling the damping characteristics by applying the current to the second damper 152 provided on the right side of the rotating frame 104.

As illustrated in FIG. 14, the rotation speed of the gantry 102 reaches v1, and from a time t2, the gantry 102 moves in the Z-axis direction under the control of the moving device 140 to scan the object.

Even when the gantry 102 moves in the Z-axis direction, the moving speed of the gantry 102 may gradually increase from the time t2 to reach a predetermined moving speed v2 and then the gantry 102 moves at the moving speed of v2. When the gantry is approaching the capturing part of the object, the moving speed of the gantry 102 may decrease so that the movement of the gantry 102 may be stopped at a time t3.

That is, in a section \textcircled{2} in which the gantry 102 moves in the Z-axis direction, because the gantry 102 moves in the Z-axis direction while the gantry 102 is rotating, the shaking occurs based on the Z-axis according to the rotation direction of the gantry 102. At the same time, the shaking occurs based on the Y-axis according to the movement of the gantry 102.

Accordingly, the controller 118 may determine the damping coefficient for attenuating the vibration in a moving section of the gantry 102 based on the damping coefficient data of the damper 150 stored in the storage 124 in advance for the section \textcircled{2}, and may apply the current to the damper 150 to operate the damper 150 according to the determined damping coefficient.

That is, the controller 118 may reduce the vibration, which is generated based on the Y-axis and the Z-axis when the gantry 102 moves while rotating, by applying the current to the first damper 151 and the second damper 152 based on the predetermined damping coefficient data.

The damping coefficients of the damper 150 for attenuating the vibration of the gantry 102 are different from each other in the section \textcircled{1} in which the rotation of the gantry 102 is accelerated and the section \textcircled{2} in which the gantry 102 is moved. The operation of the damper 150 to attenuate the vibration of the gantry 102 in each section may be controlled by the predetermined damping coefficient data.

The gantry 102 may stop the movement after the movement to the point where the object is to be captured, and the X-rays may be irradiated onto the object at the position where the gantry 102 is moved to perform the capturing. For example, the gantry 102 may operate in an axial scan mode.

As illustrated in FIG. 14, the movement of the gantry 102 is stopped at the time t3 and the X-rays are irradiated onto the object from the X-ray source 106 from a time t4 to a time t5 to perform the tomography.

Referring back to FIG. 13, when it is determined by the controller 118 that the X-rays are irradiated to the object from the X-ray source 106, the controller 118 may control the sensing device 110 to obtain the vibration data generated by the movement of the gantry 102 while the X-rays are irradiated (1020).

In a section \textcircled{3} in which the X-rays are irradiated from the X-ray source 106, the gantry 102 does not move along the Z-axis, but because the gantry 102 continues to rotate, the shaking may occur due to rotation, and the sensing device 110 may obtain the vibration data according thereto in real time.

In detail, the first sensing device 111 and the second sensing device 112 may obtain vibration data generated from the rotation of the gantry 102 and transmit the vibration data to the controller 118. The controller 118 may compare the vibration data obtained by the sensing device 110 with the predetermined reference value stored in the storage 124(1030).

As a result of the comparison, when the magnitude of the obtained vibration data exceeds the predetermined reference value, the controller 118 may determine the damping coefficient of the damper 150 to make the magnitude of the vibration data equal to or less than the predetermined reference value (1040).

That is, in the section \textcircled{1} or the section \textcircled{2} where the X-rays are not irradiated, the controller 118 may control the operation of the damper 150 according to the damping coefficient set according to the image capturing protocol to reduce the vibration generated by the movement of the gantry 102. In the section \textcircled{3} where the X-rays are irradiated, the controller 118 may determine the damping coefficient based on the vibration data obtained by the sensing device 110 in real time, and may control the operation of the damper 150 by applying the current to the damper 150 accordingly (1050).

When the X-ray irradiation of the object is completed at a time t5, the gantry 102 may move in a direction away from the object under the control of the moving device 140.

That is, in a section \textcircled{4} in which the gantry 102 moves in the Z-axis after completion of X-ray irradiation, the gantry 102 moves in the Z-axis direction while the gantry 102 rotates, as in the section \textcircled{2}, and thus the shaking occurs based on the Z-axis according to the rotation direction of the gantry 102, and at the same time, the shaking occurs based on the Y-axis according to the movement of the gantry 102.

Accordingly, the controller 118 may determine the damping coefficient for attenuating the vibration in a moving section of the gantry 102 based on the damping coefficient data of the damper 150 stored in the storage 124 in advance for the section \textcircled{4}, and may apply the current to the damper 150 to operate the damper 150 according to the determined damping coefficient.

Referring to FIG. 15, a section \textcircled{1}, in which the gantry 102 starts to rotate, accelerates to the predetermined speed v1, reaches the predetermined speed v1, and rotates at the constant speed v1, is the same as the section \textcircled{1} in FIG. 14.

In addition, as illustrated in FIG. 15, the rotation speed of the gantry 102 reaches v1, and from time t3, the gantry 102 moves in the Z-axis direction under the control of the moving device 140 to scan the object.

On the other hand, unlike in FIG. 14 illustrating that the gantry 102 moves to the capturing part of the object, stops the movement and then the X-rays are irradiated from the X-ray source 106, in FIG. 15, the X-rays are irradiated from the time t2 before the moving time t3 of the gantry 102 and the X-rays are irradiated. The gantry 102 may move in the Z-axis direction while scanning the object with the X-rays being irradiated. For example, the movement of the gantry 102 may be in a helical scan mode.

Accordingly, it is the same as in FIG. 14 that the controller 118 determines the damping coefficient for attenuating the vibration in the acceleration section of the gantry 102 based on the damping coefficient data of the damper 150 stored in the storage 124 in advance for the section \textcircled{1} and applies the current t to the damper 150 to operate the damper 150 according to the damping coefficient. However, in the section \textcircled{2}, even when the gantry 102 is moving in the Z-axis direction, the X-rays are irradiated, and thus the controller 118 controls the operation of the damper 150 based on the vibration data obtained by the sensing device 110 in real time.

That is, in the section \textcircled{2} of FIG. 15 where the X-rays are irradiated from the X-ray source 106 while the gantry 102 moves, as in section \textcircled{3} of FIG. 14, the controller 118 determines the damping coefficient based on the vibration data obtained by the sensing device 110 in real time, and accordingly, applies the current to the damper 150 to control the operation of the damper 150.

Based on the medical imaging apparatus and the method of controlling the medical imaging apparatus according to the embodiment, the shaking occurred by the rotation and movement of the gantry 102 may be reduced by controlling the operation of the damper 150 based on the predetermined damping coefficient according to the image capturing protocol for the object.

In addition, while the X-rays are irradiated to the object, the shaking occurred from the movement of the gantry 102 may be reduced by determining the damping coefficient by comparing the vibration data obtained by the sensing device 110 with the pre-stored reference value, and controlling the operation of the damper 150 according to the determined damping coefficient.

Referring back to FIG. 13, the controller 118 may store vibration attenuating data of the damper 150 in the storage 124 based on a result of controlling the operation of the damper 150, and may update data on the damping coefficient of the damper 150 pre-stored in the storage 124 (1300).

That is, it is determined and recorded how much the vibration generated by the movement of the gantry 102 is reduced by controlling the operation of the damper 150 based on the damping coefficient pre-stored in the storage 124. By calculating a new damping coefficient to be corrected according to a degree of vibration reduction of the gantry 102 and storing it in the storage 124, the stored data may be used for the operation of the damper 150 when the gantry 102 rotates and moves to scan the object later.

According to the embodiment, when performing tomography on the object, the vibration generated by rotation and movement of the medical imaging apparatus is reduced through the control of the damper 150. By updating the vibration reduction data and the damping coefficient data are in real time, it is possible to obtain accurate medical images without generation of a noise due to the vibration during tomography.

According to the medical imaging apparatus and the method of controlling the medical imaging apparatus of exemplary embodiments, there is an effect of minimizing the shaking caused by the rotation of the gantry of the mobile CT apparatus or movement of the mobile CT apparatus.

Meanwhile, the disclosed embodiments may be implemented in the form of a recording medium storing instructions that are executable by a computer. The instructions may be stored in the form of a program code, and when executed by a processor, the instructions may generate a program module to perform operations of the disclosed embodiments. The recording medium may be implemented as a computer-readable recording medium.

The computer-readable recording medium may include all kinds of recording media storing commands that can be interpreted by a computer. For example, the computer-readable recording medium may be read only memory (ROM), random access memory (RAM), a magnetic tape, a magnetic disc, flash memory, an optical data storage device, etc.

Although a few embodiments of the disclosure have been shown and described, it would be appreciated by those skilled in the art that changes may be made in these embodiments without departing from the principles and spirit of the disclosure, the scope of which is defined in the claims and their equivalents.

What is claimed is:

1. A medical imaging apparatus comprising:
a gantry that is moveable;
a moving device configured to move the medical imaging apparatus that includes the gantry;
a damper configured to attenuate vibration generated by a movement of the gantry or a movement of the medical imaging apparatus;
an inputter configured to receive an image capturing protocol for an object; and
a controller configured to:
determine a coefficient of the damper for attenuating the vibration generated by the movement of the gantry or the movement of the medical imaging apparatus corresponding to the input image capturing protocol, and
apply a current to the damper to operate the damper according to the determined coefficient of the damper.

2. The medical imaging apparatus according to claim 1, further comprising:
a storage configured to store data about predetermined coefficient of the damper corresponding to the image capturing protocol,
wherein the predetermined coefficient comprises at least one of a coefficient for attenuating the vibration generated by rotation of the gantry or a coefficient for attenuating the vibration generated as the gantry moves in a direction of scanning the object.

3. The medical imaging apparatus according to claim 1, wherein the gantry comprises a rotating frame configured to rotate about a predetermined rotation axis, and
wherein the damper is provided on the rotating frame.

4. The medical imaging apparatus according to claim 1, wherein the moving device comprises a wheel configured to move the medical imaging apparatus that includes the gantry in a direction of scanning the object, and
wherein the damper is provided on the wheel.

5. The medical imaging apparatus according to claim 3, wherein the damper is among a plurality of dampers provided in the rotating frame, and
wherein the plurality of dampers are spaced apart from each other by a predetermined distance on the rotating frame.

6. The medical imaging apparatus according to claim 4, wherein the wheel is among a plurality of wheels and the damper is among a plurality of dampers, and
wherein the plurality of dampers are provided in correspondence with the plurality of wheels.

7. The medical imaging apparatus according to claim 1, wherein the controller is configured to apply the current to the damper to change an amount of fluid flowing in the damper or to change a viscosity of the fluid for operating the damper according to the determined coefficient.

8. The medical imaging apparatus according to claim 7, wherein the damper comprises a piston channel through which the fluid flows, and
wherein the controller is configured to control opening and closing of the piston channel by applying the current to the damper to change the amount of fluid flowing in the damper.

9. The medical imaging apparatus according to claim 7, wherein the damper comprises an electromagnet configured to form a magnetic field in response to application of the current, and
wherein the controller is configured to change the viscosity of the fluid by applying the current to the electromagnet to form a magnetic field inside the damper.

10. The medical imaging apparatus according to claim 1, further comprising:
an X-ray source provided inside the gantry, and configured to irradiate X-rays; and
a sensing device configured to obtain vibration data generated by the movement of the gantry or the movement of the medical imaging apparatus while the X-ray source radiates the X-rays.

11. The medical imaging apparatus according to claim 10, wherein the controller is configured to:
determine whether the X-ray source irradiates the X-rays;
upon determining the X-ray source irradiates the X-rays, compare a magnitude of vibration according to the vibration data obtained by the sensing device with a reference value;
determine the coefficient of the damper such that the magnitude of vibration according to the vibration data obtained is equal to or less than the reference value; and
apply the current to the damper to operate the damper according to the determined coefficient.

12. The medical imaging apparatus according to claim 10, wherein the sensing device comprises at least one of an acceleration sensor, a vibration sensor, a position sensor, or a displacement sensor.

13. The medical imaging apparatus according to claim 10, wherein the gantry comprises a rotating frame configured to rotate about a predetermined rotation axis,
wherein the sensing device is among plurality sensing devices in the rotating frame of the gantry, and
wherein the plurality of sensing devices are spaced apart from each other by a predetermined distance on the rotating frame.

14. The medical imaging apparatus according to claim 10, wherein the moving device comprises a plurality of wheels configured to move the medical imaging apparatus provided with the gantry in a direction of scanning the object, and
wherein the sensing device is among a plurality of sensing devices which are provided in correspondence with the plurality of wheels.

15. The medical imaging apparatus according to claim 1, wherein the image capturing protocol comprises information about at least one of a capturing part of the object, a distance by which the gantry is to be moved to scan the object, a moving speed of the gantry, and a rotation speed of the gantry.

16. A method of controlling a medical imaging apparatus including a gantry that is moveable, an X-ray source inside the gantry configured to irradiate X-rays, and a moving device configured to move the medical imaging apparatus that includes the gantry, the method comprising:
receiving an image capturing protocol for an object to be scanned;
determining a coefficient of a damper configured to attenuate vibration generated by a movement of the gantry or a movement of the medical imaging apparatus corresponding to the input image capturing protocol; and
applying a current to the damper to operate the damper according to the determined coefficient of the damper.

17. The method according to claim 16, further comprising:
storing data about predetermined coefficient of the damper corresponding to the image capturing protocol,
wherein the predetermined coefficient comprises at least one of a coefficient for attenuating the vibration generated by rotation of the gantry or a coefficient for attenuating the vibration generated as the gantry moves in a direction of scanning the object.

18. The method according to claim 16, wherein:
the damper is controlled by applying the current to the damper to change an amount of fluid flowing in the damper or to change a viscosity of the fluid for operating the damper according to the determined coefficient.

19. The method according to claim 18, wherein changing of the amount of fluid flowing inside the damper comprises:
controlling opening and closing of a piston channel provided in the damper by applying the current to the damper to change the amount of fluid flowing in the damper.

20. The method according to claim 18, wherein changing of the viscosity of the fluid comprises:
applying the current to an electromagnet provided inside the damper to form a magnetic field inside the damper to change the viscosity of the fluid.
